(19) United States
(12) Patent Application Publication
(10) Pub. No.: US 2025/0152829 A1
(43) Pub. Date: May 15, 2025

(54) SUB-ASSEMBLY WITH FEEDBACK FOR A MEDICAMENT DELIVERY DEVICE

(71) Applicant: SHL Medical AG, Zug (CH)

(72) Inventor: Daniel Scott, Deerfield Beach, FL (US)

(21) Appl. No.: 18/838,729

(22) PCT Filed: Feb. 6, 2023
(86) PCT No.: PCT/EP2023/052772

§ 371 Date: Aug. 15, 2024

Related U.S. Application Data
(60) Provisional application No. 63/311,507, filed on Feb. 18, 2022.

(30) Foreign Application Priority Data
Apr. 19, 2022 (EP) 22168826.0

(51) Int. Cl. A61M 5/24; A61M 5/315

(52) U.S. Cl. CPC A61M 5/2429; A61M 5/31515; A61M 2205/582

(57) ABSTRACT

The present disclosure provides a sub-assembly for a medicament delivery device, the sub-assembly extending along a longitudinal axis in a longitudinal direction from a proximal end to a distal end. The sub-assembly comprises a housing and a plunger rod. The plunger rod is arranged at least partly inside the housing. The plunger rod comprises a first surface facing towards the proximal end and a component of the sub-assembly other than the plunger rod comprises a second surface facing towards the distal end, with the first surface and the second surface being arranged so that, when the plunger rod moves towards the proximal end relative to the component during medicament delivery device use, the first surface and the second surface engage to rotate the plunger rod relative to the component and then disengage so that the plunger rod rotates back relative to the component. The sub-assembly comprises a torsion spring attached at one end to the plunger rod and at the other end to the component.

### CROSS-REFERENCE TO RELATED APPLICATIONS

The present application is a U.S. National Phase Application pursuant to 35 U.S.C. § 371 of International Application No. PCT/EP2023/052772 filed Feb. 6, 2023, which claims priority to (i) U.S. Provisional Patent Application No. 63/311,507 filed Feb. 18, 2022, and (ii) European Patent Application No. 22168826.0 filed Apr. 19, 2022. The entire disclosure contents of these applications are herewith incorporated by reference into the present application.

### TECHNICAL FIELD

The invention concerns sub-assemblies for medicament delivery devices, and particularly sub-assemblies with a housing and a plunger rod.

### BACKGROUND

Some medicament delivery devices provide feedback to their users during operation, such as with the U-bracket described in WO2011/123024. While such systems can provide effective feedback, the applicant has appreciated that an alternative feedback mechanism could usefully be provided, potentially providing various advantages when compared to existing feedback mechanisms.

### SUMMARY

Reference should now be made to the appended claims.

In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which during use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which during use of the medicament delivery device is/are located closest to the dose delivery site.

Further, the terms “longitudinal”, “longitudinally”, “axially” and “axial” refer to a direction extending from the proximal end to the distal end and along the device or components thereof, typically in the direction of the longest extension of the device and/or component.

Similarly, the terms “transverse”, “transversal” and “transversally” refer to a direction generally perpendicular to the longitudinal direction.

An aspect concerns a sub-assembly for a medicament delivery device, the sub-assembly extending along a longitudinal axis in a longitudinal direction from a proximal end to a distal end, the sub-assembly comprising: a housing; and a plunger rod arranged at least partly inside the housing; wherein the plunger rod comprises a first surface facing towards the proximal end and a component of the sub-assembly other than the plunger rod comprises a second surface facing towards the distal end, with the first surface and the second surface being arranged so that, when the plunger rod moves towards the proximal end relative to the component during medicament delivery device use, the first surface and the second surface engage to rotate the plunger rod relative to the component and then disengage so that the plunger rod rotates back relative to the component (to create feedback), and wherein the sub-assembly comprises a torsion spring attached at one end to the plunger rod and at the other end to the component. This can provide a feedback such as an audible, visible or tactile feedback to a medicament delivery device user.

Optionally, the component is the housing. Optionally, the housing comprises an outer housing and a rear cap, and the rear cap comprises the second surface.

Optionally, the second surface is on a protrusion. Optionally, the protrusion extends towards the longitudinal axis. Optionally, the first surface is on a second protrusion. Optionally, the second protrusion extends away from the longitudinal axis.

Optionally, at least part of at least one of the first surface and the second surface is angled relative to a plane perpendicular to the longitudinal axis.

Optionally, the first surface and/or the second surface comprises a surface (79) that is parallel to the longitudinal axis.

Optionally, the at least part of at least one of the first surface and the second surface is angled relative to a plane perpendicular to the longitudinal axis at an angle of between 10 and 85 degrees, particularly 30 to 70 degrees, most particularly 45 to 60 degrees.

Optionally, at least part of the first surface is angled relative to a plane perpendicular to the longitudinal axis. Optionally, the sub-assembly comprises a feedback surface angled relative to a plane perpendicular to the longitudinal axis, wherein the feedback surface is spaced apart in a circumferential direction relative to the longitudinal axis from a distal end of the first surface. Optionally, the feedback surface is parallel to the longitudinal axis.

Optionally, at least part of the second surface is angled relative to a plane perpendicular to the longitudinal axis. Optionally, the sub-assembly comprises a feedback surface angled relative to a plane perpendicular to the longitudinal axis, wherein the feedback surface is spaced apart in a circumferential direction relative to the longitudinal axis from a proximal end of the first surface. Optionally, the feedback surface is parallel to the longitudinal axis.

Optionally, the plunger rod comprises the feedback surface. Optionally, the component is rotationally locked relative to the housing.

Optionally, a spring is arranged between the plunger rod and the housing, and the spring is configured to move the plunger rod towards the proximal end during medicament delivery. Optionally, the spring and the torsion spring are the same component. Alternatively, the torsion spring is a first spring and the spring is a second spring.

An aspect concerns a medicament delivery device comprising a sub-assembly as described above. Optionally, the medicament delivery device is an autoinjector.

An aspect concerns a method of creating feedback in a medicament delivery device, the method comprising the following steps in the following order: moving a plunger rod in a proximal direction relative to another component so that a proximally facing surface of the plunger rod engages a distally facing surface of the another component, thereby rotating the plunger rod in a first direction relative to the another component; moving the plunger rod further in the proximal direction relative to the another component so that the proximally facing surface of the plunger rod disengages the distally facing surface of the another component and subsequently rotates the plunger rod in a second direction, thereby creating feedback. Typically, the first direction and the second direction are opposite directions.

An aspect concerns a sub-assembly for a medicament delivery device, the sub-assembly extending along a longitudinal axis in a longitudinal direction from a proximal end to a distal end, the sub-assembly comprising: a housing; and a plunger rod arranged at least partly inside the housing; wherein the plunger rod comprises a first engagement portion and a component of the sub-assembly other than the plunger rod comprises a second engagement portion, wherein the first engagement portion and the second engagement portion are arranged so that, when the plunger rod moves relative to the component during medicament delivery device use, the first engagement portion engages the second engagement portion so that the plunger rod rotates relative to the component and then disengages the second engagement portion so that the plunger rod rotates back relative to the component. Optionally, the sub-assembly comprises a torsion spring attached at one end to the plunger rod and at the other end to the component. Optionally, the first engagement portion comprises a proximally facing surface. Optionally, the second engagement portion comprises a distally facing surface. Optionally, the first engagement portion and/or the second engagement portion is a protrusion.

Generally, all terms used in the claims are to be interpreted according to their ordinary meaning in the technical field, unless explicitly defined otherwise herein. All references to a/an/the element, apparatus, member, component, means, etc. are to be interpreted openly as referring to at least one instance of the element, apparatus, member component, means, etc., unless explicitly stated otherwise.

### BRIEF DESCRIPTION OF THE DRAWINGS

Embodiments of the present disclosure will now be described by way of example only and with reference to the following accompanying drawings.

FIG. 1 shows a perspective view of the components of an example sub-assembly.

FIG. 2 shows a close-up of the proximal end of the rear cap of the sub-assembly of FIG. 1.

FIGS. 3 to 6 show side views of another example sub-assembly in various stages during medicament delivery.

FIG. 7 shows a perspective view of the torsion spring of the example of FIG. 3.

FIG. 8 shows a perspective view of the plunger rod of the example of FIG. 3.

FIGS. 9 and 10 show the protrusions of the plunger rods of FIGS. 1 and 8 respectively. FIG. 11 shows a perspective view of an example autoinjector that could comprise any of the sub-assemblies depicted in the previous figures.

FIGS. 12 to 15 show alternative shapes for the protrusions of the plunger rod and the rear cap.

### DETAILED DESCRIPTION

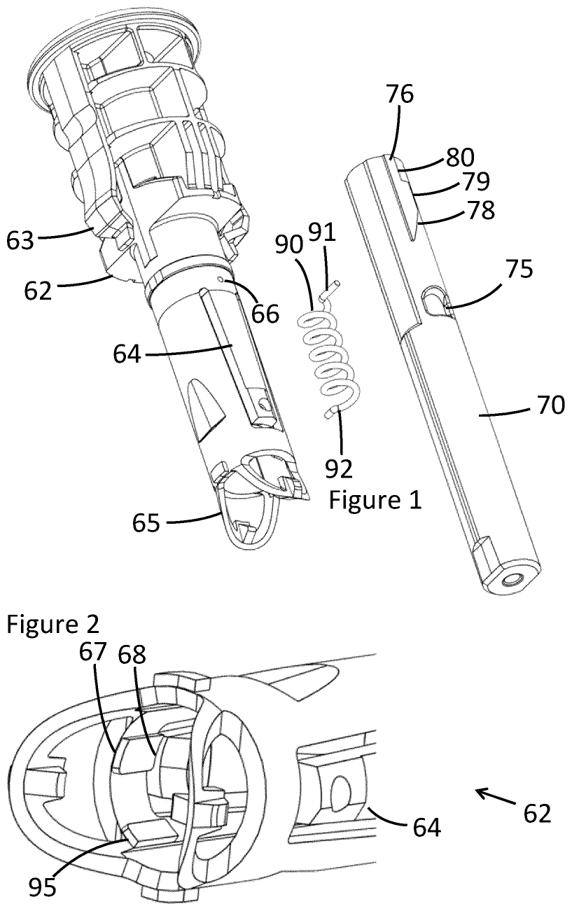

FIG. 1 shows the separate components of a sub-assembly for a medicament delivery device. The sub-assembly comprises a housing (in this case a rear cap 62), a plunger rod 70 and a torsion spring 90. The torsion spring 90 is attached at one end to the plunger rod 70 and at the other end to the rear cap 62. In this example, a distal end 91 of the torsion spring 90 is attached in a hole 66 of the rear cap 62, and a proximal end 92 of the torsion spring 90 is attached in a hole (not shown) in the plunger rod 70. Optionally, the torsion spring 90 has a straight section at each end, with the straight section optionally extending directly away from the axis as shown in FIG. 1; this can help attach the spring to the other components (in this case the rear cap 62 and the plunger rod 70).

The plunger rod 70 comprises a proximally facing surface (first surface) facing towards the proximal end and a component of the sub-assembly other than the plunger rod (in this case the housing, and more particularly the rear cap 62) comprises a distally facing surface (second surface) facing towards the distal end. In the example shown in FIG. 1, the proximally facing surface is a surface 78 of a protrusion 76, where the protrusion extends from an outer surface of the plunger rod 70. As can be seen in FIG. 2 (which shows a close-up of the proximal end of the rear cap 62), the distally facing surface is a surface 68 of a protrusion 67, where the protrusion extends from an inner surface of the rear cap 62. Optionally, as shown in FIG. 2, a second protrusion 95 can be provided on the rear cap; this can help prevent over-rotation of the plunger rod.

During use of a medicament delivery device comprising the sub-assembly, the plunger rod moves relative to the component (in this case the rear cap), resulting in the proximally facing surface and the distally facing surface engaging to rotate the plunger rod relative to the component, and then subsequently disengaging so that the plunger rod rotates back relative to the component, which can create feedback (e.g. audible, visible and/or tactile feedback).

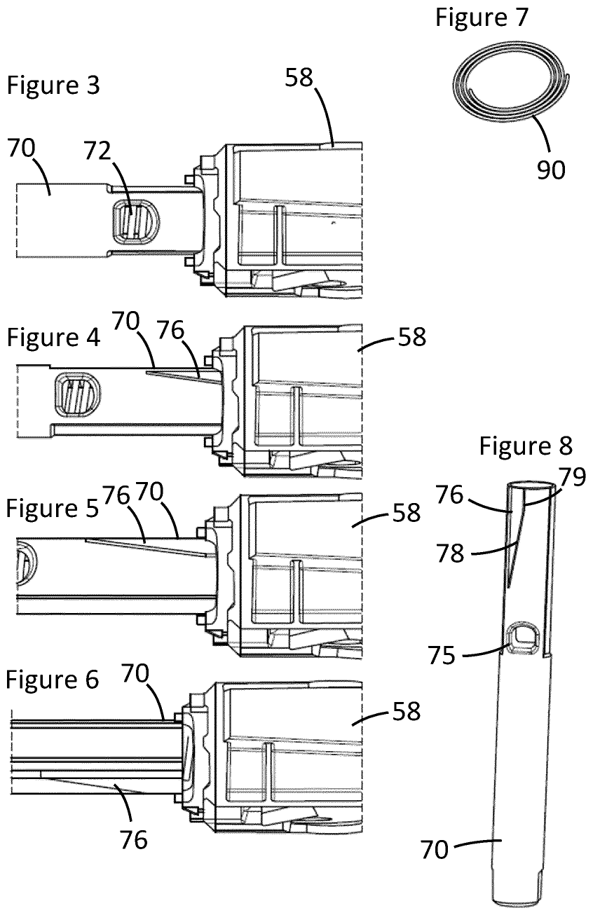

A second example sub-assembly is shown in FIGS. 3 to 8. In this example, the protrusion 76 is a different shape and a differently-shaped torsion spring 90 is used, but otherwise, this example is largely the same as the example in FIGS. 1 and 2.

FIG. 11 shows an example of an autoinjector 10 which could comprise any of the sub-assemblies depicted in the figures. For reference, a longitudinal axis 12, an axial direction 13, a proximal end 14, a distal end 15, a circumferential direction 16 and a radial direction 17 are shown. The autoinjector comprises a housing comprising an outer housing 30 (with an optional window 32) and a rear cap 62, along with a needle guard 40 (more generally a medicament delivery member guard). A typical autoinjector would also include a powerpack to drive the plunger rod. The powerpack could provide power using a power source such as a spring, a battery or compressed gas, for example. The rear cap 62 can be part of the powerpack, and could house the power source. A medicament container such as a syringe or a cartridge would be provided, and a medicament delivery member, such as a needle or a jet injector, would be provided. Although not essential, the powerpack would typically be locked until the device is activated for use, for example by the rotator 58 shown in the figures being rotated by a medicament delivery member guard (e.g. needle guard 40), or by another component such as a button. A medicament delivery container carrier such as a syringe carrier could also be provided, although the medicament delivery container could be supported by one or more other components instead, such as by a housing. Although an autoinjector is described, the sub-assembly could also be used in other injectors or other medicament delivery devices more generally, including non-automatic medicament delivery devices such as pen injectors, and components such as the rear cap, the powerpack, the rotator and the medicament delivery member guard are therefore optional.

The particular structure, shape and attachment points of the torsion springs as shown in the examples are optional and could be varied, with the torsion spring attached in grooves rather than in holes (e.g. hole 66 in FIG. 1), or attached by glue, for example.

Optionally, the spring of the powerpack and the torsion spring could be the same spring, or alternatively could be two separate springs. In the example where they are the same spring, energy could be stored for medicament delivery by compressing the spring, and energy can be stored for providing the feedback by twisting the spring as described above.

The method of operation of the sub-assembly will now be described in more detail with reference to the second example sub-assembly as depicted in FIGS. 3 to 8. In FIG. 3, the plunger rod 70 has already been released and is moving towards the proximal end of the medicament delivery device (i.e. moving in the proximal direction relative to the rear cap). As the respective surfaces 67, 76 of the plunger rod and the rear cap have not yet engaged one another, the plunger rod has not yet rotated relative to the rear cap.

In FIG. 4, the respective surfaces of the plunger rod and the rear cap have now engaged one another and the plunger rod has started to rotate relative to the rear cap. Since the torsion spring is attached at one end to the rear cap and at the other end to the plunger rod, the torsion spring is elastically deformed by the rotation of the plunger rod relative to the rear cap, thereby transferring some of the energy from the power source that is driving the plunger rod to the torsion spring.

In FIG. 5, the plunger rod has now rotated to its full extent relative to the rear cap. As the plunger rod continues to move in the proximal direction relative to the rear cap, the plunger rod disengages from the rear cap, which allows the energy built up in the torsion spring to rotate the plunger rod back in the opposite direction to the position shown in FIG. 6. This rotation can be rapid, allowing for feedback (in particular an audible click) due to an impact with another component.

The particular structure of the engagement portion of the plunger rod and the corresponding engagement portion of the rear cap will now be dealt with in more detail. In the examples in the figures, two different options are given, both with a protrusion on the plunger rod and a corresponding protrusion on the rear cap. In these cases, the protrusion on the plunger rod could be considered to be the engagement portion of the plunger rod, and the protrusion of the rear cap could be considered to be the engagement portion of the rear cap. Further alternative structures for the engagement portions will be described later.

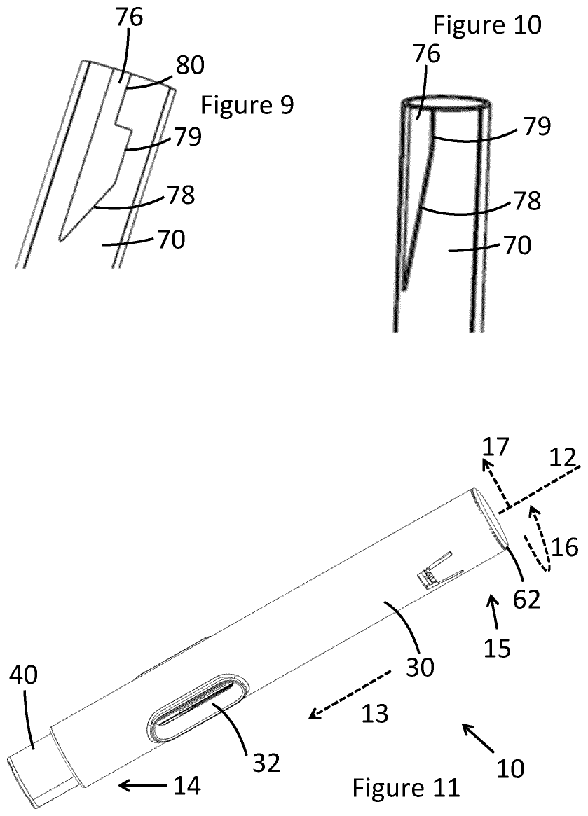

In the first example as shown in FIGS. 1 and 2, a protrusion 76 with the shape shown in FIG. 9 is provided. The protrusion comprises a surface 78 (rotation surface), a surface 79 (holding surface) and a surface 80 (impact surface). The surface 78 is adjacent to the surface 79, so that, during use, the surface 68 of the protrusion 67 moves directly from abutting the surface 78 to abutting the surface 79. A further surface 80 is spaced apart from the surface 79 (it is offset in the circumferential direction). This gap between the surface 79 and the surface 80 allows for acceleration of the protrusion 76 relative to the protrusion 67, thereby allowing the impact of the protrusion 67 on the surface 80 to create feedback (in particular audible feedback). The surface 78 is a proximally facing surface, and is angled relative to the longitudinal axis—this angle allows the engagement between the protrusion 67 and the protrusion 76 to rotate the plunger rod relative to the rear cap. The surface 79 is optional, but including a surface 79 or varying the length of the surface 79 can be used to regulate the point during device use at which the energy built up in the torsion spring is released. The surface 79 is parallel to the longitudinal axis, though this is optional. The surface 80 is parallel to the longitudinal axis, though this is optional.

In the second example as shown in FIGS. 3 to 8, a protrusion 76 with the shape shown in FIG. 10 is provided. The protrusion 76 comprises a surface 78 and a surface 79 as in FIG. 9 (albeit with slightly different dimensions). The surface 80 of FIG. 9 is not included; instead, a surface for the protrusion 67 to hit could be provided on another component. More generally, a surface for the protrusion 67 to hit is also optional, as the feedback could instead be tactile or visual, for example with the protrusion 76 visible through a window in an outer housing after the protrusion 76 has passed the surface 79.

Although the examples herein provide the surfaces that engage one another on protrusions, this is optional. For example, one of the surfaces could be on a protrusion, with the other surface being the wall of a slot (for example a helical slot), or one or more of the surfaces could be created by a discontinuity such as a step.

In the examples shown herein, the engagement portions (the surfaces that engage one another) are on the rear cap 62 and the plunger rod 70. Instead of being on the rear cap, the engagement portion could be on another component, such as on another part of the housing (e.g. an outer housing), a syringe carrier, or a rotator. A rotator in medicament delivery devices is typically rotatable relative to other components such as the housing and the plunger rod only at particular points during medicament delivery device use, and is rotationally restricted during other points, for example during medicament delivery. As such, the rotator can be used to house the engagement portion if the rotational state of the rotator is taken into account.

In the examples shown herein, the plunger rod 70 is held in place before use within the rear cap 62 by the arm 64 of the rear cap 62 interacting with the hole 75 of the plunger rod 70 (see FIG. 1), although other mechanisms for holding the plunger rod in place prior to use could also be provided).

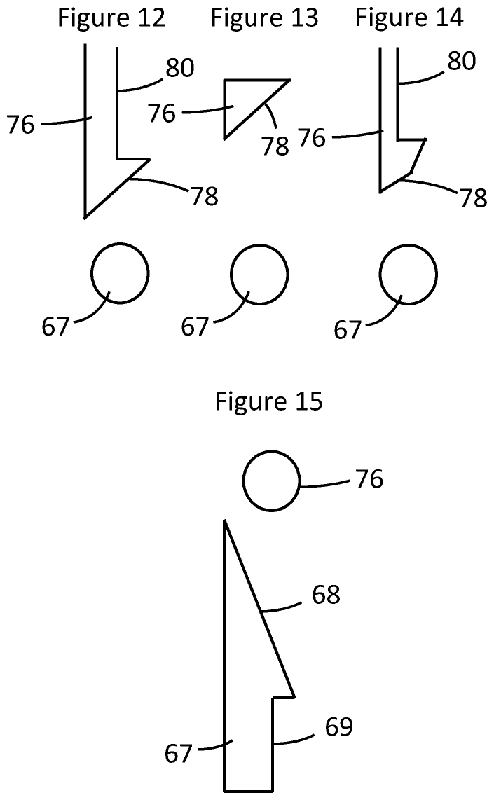

Various other modifications could be made to the example protrusions shown in FIGS. 9 and 10. For example, the protrusion of FIG. 9 or the protrusion of FIG. 10 could be replaced by a protrusion with a shape as shown in one of FIGS. 12 to 14. A protrusion 67 of the housing (e.g. rear cap) is also shown for context. In FIGS. 12 to 14, the protrusion 67 of the housing is circular. In general, the protrusion 67 could be various shapes, including circular, triangular, square, or an irregular shape.

In the examples provided above, the protrusion 67 of the rear cap has surfaces that are parallel to the longitudinal axis (the surface 68) and perpendicular to the longitudinal axis, and the only surface that is angled relative to the longitudinal axis is the surface 78 of the plunger rod. The two engagement portions could be switched, with the protrusion 76 provided on the rear cap and the protrusion 67 provided on the rear cap. An example where the angled surface is on the housing (e.g. rear cap), rather than on the plunger rod, is shown in FIG. 15. In this example, the surface 68 is angled in the same way as the surface 78 of the plunger rod in the examples in FIGS. 12 to 14, but with the surface 68 angled towards the distal end rather than towards the proximal end. The protrusion 76 of the plunger rod in this example is circular, but could again be various other shapes, including triangular, square, or an irregular shape. A surface 69 (impact surface), which is analogous to the surface 80 of the other depicted examples but faces in the distal direction rather than in the proximal direction, is also optionally provided.

It is envisioned that two protrusions 76 would be provided on the plunger rod, with two corresponding protrusion 67 on the rear cap. Typically, the two protrusions 76 would be opposite each other relative to the longitudinal axis, and the two protrusions 67 would also be opposite to each other relative to the longitudinal axis, though this is optional and a different spacing could also be used. Alternatively, one, three or more protrusions 76 could be provided, with the protrusions optionally equally spaced around the axis. Generally, a protrusion 67 on the rear cap would be provided for each protrusion 76 on the plunger rod.

Typically, at least one of the protrusion on the rear cap and the protrusion on the plunger rod comprises a surface (such as the surface 78) that is angled relative to a plane perpendicular to the longitudinal axis; that is, a surface that extends in a direction with an angle of greater than zero degrees and less than 90 degrees relative to a plane perpendicular to the longitudinal axis, for example between 10 and 85 degrees. Preferably, the angle is between 30 and 70 degrees, or between 45 and 60 degrees. The examples of the surface 78 shown in the figures all have a constant angle, but the angle could also vary, and could for example be curved when viewed from the angle shown in FIG. 9 (i.e. perpendicular to the longitudinal axis with the longitudinal axis behind the protrusion). As shown in FIG. 14, the surface 78 could also have two sections at different angles.

In the example in FIGS. 3 to 8, the protrusion 76 comprises a surface 79 facing perpendicular to the axis in addition to the proximally facing surface 78. This surface 79 is optional, but can be useful to adjust the timing of feedback.

The particular shape of the rear cap 62 shown in the figures can be varied, and various other optional rear cap features, such as a powerpack transport locking arm 63, a pre-activation locking arm 64 and syringe support arms 65, are shown but are not described in detail as they are not directly relevant to providing feedback. The plunger rod is shown inside the rear cap in the depicted examples (i.e. the plunger rod is arranged at least partly inside the housing—at least partly inside the rear cap of the housing in these examples), with the protrusion of the plunger rod extending from an outer surface of the plunger rod and the protrusion of the rear cap extending from an inner surface of the rear cap, but this could be reversed, with the protrusion of the plunger rod on an inner surface of the plunger rod and the protrusion of the rear cap extending from an outer surface of the rear cap (for example from an outer surface of a tubular extension of the rear cap that extends inside the distal end of the plunger rod).

In this application, it is primarily envisioned that the feedback would be provided at the beginning or end of delivery, but the time of the feedback could be adjusted by adjusting the relative locations of the features described above. Optionally, multiple instances of feedback could be provided, for example with a selection from a start of delivery feedback, an end of delivery feedback, and ‘continuous’ feedback during delivery (i.e. multiple closely spaced clicks providing multiple instances of feedback). Two (or more) instances of feedback could be achieved by two (or more) protrusions on the plunger rod at different axial positions. The feedback could be audible, tactile and/or visible, for example.

The drug delivery devices described herein can be used for the treatment and/or prophylaxis of one or more of many different types of disorders. Exemplary disorders include, but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary drugs that could be included in the drug delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)). Pharmaceutical formulations including, but not limited to, any drug described herein are also contemplated for use in the drug delivery devices described herein, for example pharmaceutical formulations comprising a drug as listed herein (or a pharmaceutically acceptable salt of the drug) and a pharmaceutically acceptable carrier. Pharmaceutical formulations comprising a drug as listed herein (or a pharmaceutically acceptable salt of the drug) may include one or more other active ingredients, or may be the only active ingredient present.

Various modifications to the embodiments described are possible and will occur to those skilled in the art without departing from the invention which is defined by the following claims.

1-15. (canceled)

16. A sub-assembly for a medicament delivery device, the sub-assembly extending along a longitudinal axis in a longitudinal direction from a proximal end to a distal end, the sub-assembly comprising:
a housing; and
a plunger rod arranged at least partly inside the housing;
wherein the plunger rod comprises a first surface facing towards the proximal end and a component of the sub-assembly other than the plunger rod comprises a second surface facing towards the distal end, with the first surface and the second surface being arranged so that, when the plunger rod moves towards the proximal end relative to the component during medicament delivery device use, the first surface and the second surface engage to rotate the plunger rod relative to the component and then disengage so that the plunger rod rotates back relative to the component, and
wherein the sub-assembly comprises a torsion spring attached at one end to the plunger rod and at the other end to the component.

17. The sub-assembly of claim 16, wherein the component is the housing.

18. The sub-assembly of claim 16, wherein the housing comprises an outer housing and a rear cap, and the rear cap comprises the second surface.

19. The sub-assembly of claim 16, wherein at least part of at least one of the first surface and the second surface is angled relative to a plane perpendicular to the longitudinal axis.

20. The sub-assembly of claim 19, wherein the at least part of at least one of the first surface and the second surface is angled relative to a plane perpendicular to the longitudinal axis at an angle of between 10 and 85 degrees.

21. The sub-assembly of claim 19, wherein at least part of the first surface is angled relative to a plane perpendicular to the longitudinal axis.

22. The sub-assembly of claim 21, wherein the sub-assembly comprises a feedback surface angled relative to a plane perpendicular to the longitudinal axis, wherein the feedback surface is spaced apart in a circumferential direction relative to the longitudinal axis from a distal end of the first surface.

23. The sub-assembly of any of claim 19, wherein at least part of the second surface is angled relative to a plane perpendicular to the longitudinal axis.

24. The sub-assembly of claim 23, wherein the sub-assembly comprises a feedback surface angled relative to a plane perpendicular to the longitudinal axis, wherein the feedback surface is spaced apart in a circumferential direction relative to the longitudinal axis from a proximal end of the first surface.

25. The sub-assembly of claim 24, wherein the plunger rod comprises the feedback surface.

26. The sub-assembly of claim 16, wherein the component is rotationally locked relative to the housing.

27. The sub-assembly of claim 16, wherein a spring is arranged between the plunger rod and the housing, and the spring is configured to move the plunger rod towards the proximal end during medicament delivery.

28. The sub-assembly of claim 27, wherein the spring and the torsion spring are the same component.

29. The sub-assembly of claim 27, wherein the torsion spring is a first spring and the spring is a second spring.

30. A medicament delivery device comprising the sub-assembly of claim 16.
